(19) United States
(12) Patent Application Publication
(10) Pub. No.: US 2025/0114060 A1
(43) Pub. Date: Apr. 10, 2025

(54) RADIATION IMAGING APPARATUS, RADIATION IMAGING SYSTEM, RADIOGRAPHIC IMAGE PROCESSING APPARATUS, METHOD OF CONTROLLING RADIATION IMAGING APPARATUS, AND MEDIUM

(71) Applicant: CANON KABUSHIKI KAISHA, Tokyo (JP)

(72) Inventor: KAZUAKI UMEKAWA, Tokyo (JP)

(21) Appl. No.: 18/911,534

(22) Filed: Oct. 10, 2024

(30) Foreign Application Priority Data
Oct. 10, 2023 (JP) 2023-175218

(51) Int. Cl. A61B 6/58

(52) U.S. Cl. CPC A61B 6/585

(57) ABSTRACT

A radiation imaging apparatus includes: an image data acquisition unit which acquires a radiographic image that is related to a photography mode selected from a plurality of photography modes; an offset correction data acquisition unit which acquires an offset correction data corresponding to each of the plurality of photography modes; an offset correction unit which corrects the radiographic image with use of acquired offset correction data; a determination unit which determines, out of the plurality of photography modes, a group of photography modes for which common offset correction data shared with one another is usable, and selects, out of offset images corresponding to respective photography modes in the group, an offset image to be used in offset correction; and an update unit which updates, in order to execute offset correction of the photography modes in the group, the selected offset image to a corresponding offset image.

### BACKGROUND OF THE INVENTION

### Field of the Invention

The present disclosure relates to a radiation imaging apparatus, a radiation imaging system, a radiographic image processing apparatus, a method of controlling a radiation imaging apparatus, and a medium.

### Description of the Related Art

A radiation imaging apparatus and a radiation imaging system in which an object is irradiated with radiation from a radiation generation apparatus, and a radiographic image is acquired from an intensity distribution of radiation transmitted through the object have been productized. In the radiation imaging apparatus and the radiation imaging system, a sharp radiographic image is acquired by digitizing the intensity distribution of the radiation and executing image processing on a radiographic image acquired as a result of the digitization.

The radiographic image generated by the radiation imaging apparatus includes not only a component corresponding to incident radiation but also a component corresponding to a dark charge. In order to acquire a suitable radiographic image, removal of the component corresponding to a dark charge is required. As a method for this removal, there has been known a method (offset correction) in which an image acquired with the radiation imaging apparatus being irradiated with no radiation (a so-called offset image) is subtracted from the radiographic image. A quantity of generated dark charges depends on an internal temperature of the radiation imaging apparatus. Accordingly, when there is a difference between distribution of the temperature inside the radiation imaging apparatus at a time of acquisition of the offset image and the internal temperature distribution at a time of acquisition of the radiographic image, removal of an offset component in a correct manner may not be executable. In acquisition of the radiographic image, the offset image to be used for the subtraction is required to be acquired, or updated, appropriately.

Various proposals have been made with respect to acquisition of such an offset image, or update of the offset image. For example, in Japanese Patent Application Laid-Open No. 2003-190126, there is described a method in which, when a new offset image is acquired in one mode out of a plurality of radiography modes, offset information of the other modes is calculated and updated based on the acquired offset image. In Japanese Patent Application Laid-Open No. 2017-006331, there is described a method in which an offset image is acquired in accordance with an order and a frequency of acquisition of an offset image which are set based on photographing conditions of a plurality of radiography modes.

The method as described in Japanese Patent Application Laid-Open No. 2003-190126 takes time, when the number of the plurality of radiography modes is large, to calculate offset information for all of the other modes based on the acquired offset image. The method accordingly has a possibility that, depending on the number of radiography modes, update of offset information of all radiography modes prolongs a period in which photographing is inexecutable. In the method as described in Japanese Patent Application Laid-Open No. 2017-006331, the order of acquiring offset images is determined. Accordingly, when the number of the plurality of radiography modes is large, an offset image is acquired and updated infrequently for a radiography mode that is in a later place in the order of acquisition. As a result, in a case in which a time available for acquisition of offset images is limited, appropriate offset correction is inexecutable depending on which radiography mode is used, and inappropriate offset correction may greatly affect image quality.

### SUMMARY OF THE INVENTION

The present disclosure has been made in view of the situation described above, and one of objects of the present disclosure is to provide a radiation imaging apparatus capable of acquiring pieces of offset information corresponding to a large number of radiography modes in a shorter time.

In order to solve the problem described above, a radiation imaging apparatus according to one embodiment of the present disclosure includes: an image data acquisition unit configured to acquire radiographic image data that is related to a photography mode selected from a plurality of photography modes for acquiring radiographic images of a plurality of types; an offset correction data acquisition unit configured to acquire offset correction data corresponding to each of the plurality of photography modes; an offset correction unit configured to correct the radiographic image data with use of the acquired offset correction data; a determination unit configured to determine, out of the plurality of photography modes, photography modes for which common offset correction data shared with one another is usable, and select, out of pieces of offset correction data corresponding to the respective determined photography modes, a piece of offset correction data to be used by the offset correction unit in correction of the determined photography modes; and an update unit configured to control the offset correction data acquisition unit so that, in order to update offset correction data to be used for the photography modes determined to be suitable for shared use of the common offset correction data, offset correction data corresponding to the selected offset correction data is acquired.

In the radiation imaging apparatus according to one embodiment of the present disclosure, it is possible to acquire pieces of offset information corresponding to a large number of radiography modes in a shorter time.

Further features of the present disclosure will become apparent from the following description of exemplary embodiments with reference to the attached drawings.

### BRIEF DESCRIPTION OF THE DRAWINGS

FIG. 1 is a diagram for illustrating a configuration example of a radiation imaging system.

FIG. 2 is a diagram for illustrating a configuration example of a radiation detection unit in a radiation imaging apparatus.

FIG. 3 is a flow chart for determining offset correction data of the radiation imaging apparatus.

FIG. 4 is a table for showing photography mode information of the radiation imaging apparatus.

FIG. 5 is a flow chart for illustrating offset image update processing of the radiation imaging apparatus.

FIG. 6 is a diagram for illustrating a configuration example of a radiographic image processing apparatus according to a modification example.

### DESCRIPTION OF THE EMBODIMENTS

An exemplary mode for carrying out the present disclosure is described in detail below as an embodiment with reference to the accompanying drawings. Matters described in the following embodiment, which include dimensions, materials, or relative positions of components, are freely set, and can be changed depending on various conditions or configurations to which the present disclosure is applied. In the drawings, the same reference symbols are used to denote components that are the same as one another or functionally similar to one another among the drawings.

In the following embodiments, a radiation imaging system using an X-ray as an example of radiation is described. However, the radiation imaging system according to the present disclosure may use other types of radiation. The term “radiation” here may include, for example, electromagnetic radiation such as an X-ray and a γ-ray, as well as particle radiation such as an α-ray, a β-ray, a particle ray, a photon ray, a heavy ion ray, and a meson ray.

### Embodiment

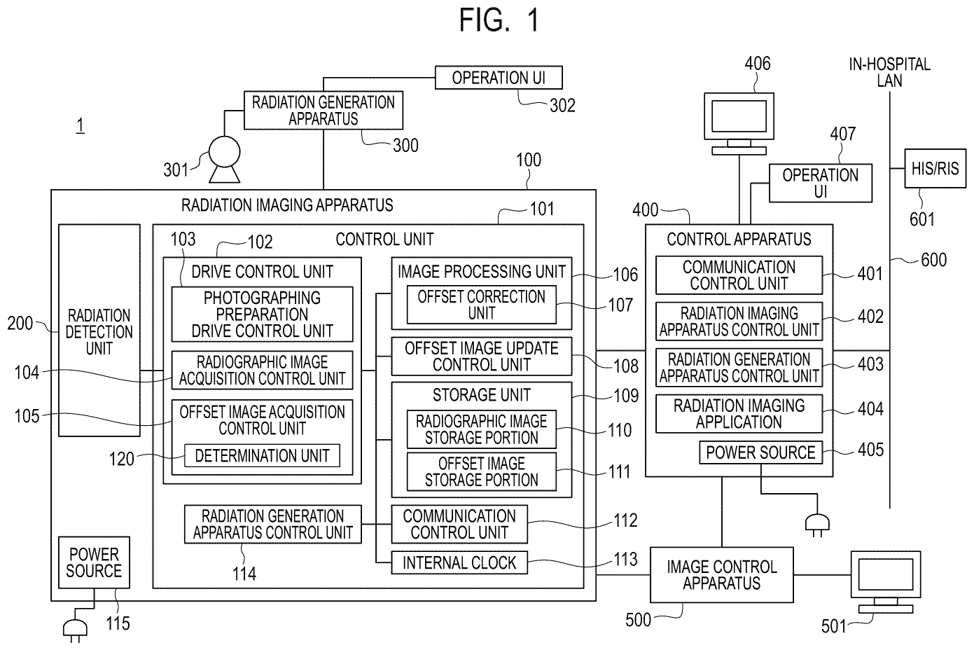

A radiation imaging apparatus, a radiation imaging system, a radiographic image processing apparatus, a method of controlling a radiation imaging apparatus, and an image processing method according to an embodiment of the present disclosure are described below with reference to FIG. 1 to FIG. 5. FIG. 1 is illustration of an example of a configuration of a radiation imaging system 1 according to this embodiment. The radiation imaging system 1 includes a radiation imaging apparatus 100, a radiation generation apparatus 300, a radiation source 301, a control apparatus 400, and an image control apparatus 500. The radiation imaging apparatus 100 includes a radiation detection unit 200. The radiation generation apparatus 300 controls the radiation source 301 which radiates radiation. The control apparatus 400 includes a radiation imaging application 404 which controls the radiation imaging apparatus 100 and the radiation generation apparatus 300, and which is capable of collection and display of a picked-up image from the radiation imaging apparatus 100 as well as reception of photographing order and registration of photographing information. The image control apparatus 500 outputs, to the control apparatus 400, a picked-up image output from the radiation imaging apparatus 100.

The control apparatus 400 is connected to an in-hospital network 600 configured from, for example, a local area network (LAN). A radiology information system (RIS) which is a radiology information system 601, or a hospital information system (HIS) which is a hospital information system 601, is connected to the in-hospital network 600. The control apparatus 400 and the HIS/RIS 601 can hold communication to and from each other, and the communication enables exchanges of a photographing order to take a radiographic image, photographing information including, for example, patient information, and photographed image data itself.

The radiation imaging apparatus 100 includes the radiation detection unit 200 which detects radiation and generates image data, a control unit 101 which controls photographing and communication operation, and a power source 115. The control unit 101 includes a drive control unit 102, an image processing unit 106, an offset image update control unit 108, a storage unit 109, a communication control unit 112, an internal clock 113, and a radiation generation apparatus control unit 114. The drive control unit 102 controls driving of the radiation detection unit 200, and acquisition of a radiographic image and an offset image. The image processing unit 106 executes image processing on an image acquired from the radiation detection unit 200. The offset image update control unit 108 controls timing of offset image update. The storage unit 109 stores acquired image data. The communication control unit 112 controls communication to and from the control apparatus 400, and communication to and from the radiation generation apparatus 300. The internal clock 113 acquires a photographed time, an elapsed time, and the like. The radiation generation apparatus control unit 114 controls irradiation timing based on an irradiation signal of radiation of the radiation generation apparatus 300.

The control unit 101 reads, for example, a program stored in the storage unit 109 and executes overall control of the radiation imaging apparatus 100 based on the program. The control of the radiation imaging apparatus 100 may be executed by a control signal generation circuit such as an ASIC, or may be implemented by a combination of a program and a control circuit.

The radiation generation apparatus 300 includes an operation user interface (UI) 302 for operating the radiation generation apparatus 300. Use of the operation UI 302 enables a user to set a radiation irradiation condition and give an instruction about radiation irradiation. This embodiment is configured so that information can be exchanged between the radiation generation apparatus 300 and the radiation imaging apparatus 100 through a dedicated signal line. This dedicated signal line is used for exchanges of synchronization signals between the radiation generation apparatus 300 and the radiation imaging apparatus 100, with regards to notification of start and end of radiation irradiation, notification of timing at which radiation irradiation is executable, and the like.

The control apparatus 400 includes a communication control unit 401, a radiation imaging apparatus control unit 402, a radiation generation apparatus control unit 403, and a power source 405. The communication control unit 401 controls communication to and from the radiation imaging apparatus 100, to and from the radiation generation apparatus 300, and to and from the in-hospital LAN. The radiation imaging apparatus control unit 402 controls image acquisition timing, an image acquisition condition, and the like of the radiation imaging apparatus 100. The radiation generation apparatus control unit 403 controls a radiation irradiation condition and the like of the radiation generation apparatus 300. The power source 405 functions as a main power source of the control apparatus 400.

The control apparatus 400 also includes the above-mentioned radiation imaging application 404 which controls the radiation imaging apparatus 100 and the radiation generation apparatus 300, and which is capable of acquisition and display of a picked-up image from the radiation imaging apparatus 100 as well as reception of photographing order and registration of photographing information. A display unit 406 for displaying the picked-up image and the photographing information and an operation UI (a keyboard, a mouse, and the like) 407 for operating the radiation imaging application 404 may be connected to the control apparatus 400.

Exchanges of information are executable between the control apparatus 400 and the radiation imaging apparatus 100, and between the control apparatus 400 and the radiation generation apparatus 300. Those exchanges of information are enabled, for example, by one or a plurality of measures out of cable connection communication using such standards as RS232C, USB, and Ethernet, a dedicated signal line, and wireless communication. Control communication including, for example, acquisition of image data, setting of an image acquisition condition, and acquisition of information about an apparatus state, is executed between the control apparatus 400 and the radiation imaging apparatus 100. Control communication including, for example, setting of a radiation irradiation condition, acquisition of information about an apparatus state, and acquisition of actual irradiation information, is executed between the control apparatus 400 and the radiation generation apparatus 300.

The image control apparatus 500 executes image processing of an image transferred from the radiation imaging apparatus 100, and transfers the processed image to the control apparatus 400. A display unit 501 is further connected to the image control apparatus 500, and the display unit 501 is used when, for example, the control apparatus 400 is down, to display an image output from the radiation imaging apparatus 100.

Although being described as separate components in the example, the control apparatus 400 and the image control apparatus 500 may be configured as an integrated component. The control apparatus 400 and the image control apparatus 500 are each configurable from a computer provided with a processor and a memory. The control apparatus 400 and 500 may be configured from general computers, or from computers specific to a radiograph system. The control apparatus 400 and 500 may be integrated with the display unit 406 and the operation UI 407 into a single personal computer (PC).

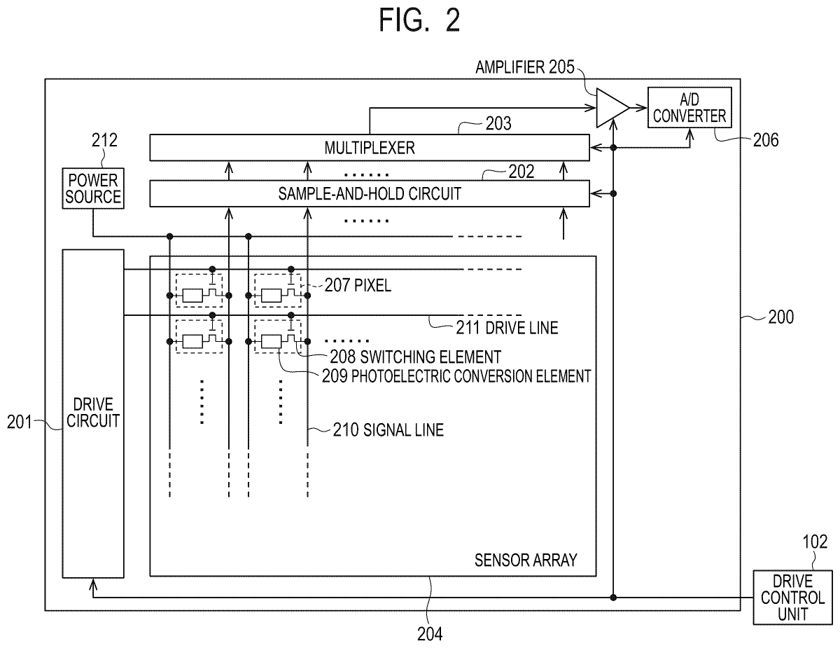

The radiation detection unit 200 is described next. FIG. 2 is illustration of an example of a configuration of the radiation detection unit 200. The radiation detection unit 200 includes a sensor array 204 built from a plurality of pixels arranged in a pattern of a two-dimensional array so as to form a plurality of rows and a plurality of columns. Each pixel 207 in the sensor array 204 is configured from a switching element 208 and a photoelectric conversion element 209 which are, for example, TFTs. A phosphor, for example, is provided in each pixel 207. Radiation incident on the radiation detection unit 200 is converted by the phosphor into visible light, the visible light resulting from the conversion enters the photoelectric conversion element 209 of each pixel, and an electric charge depending on the visible light is generated in each photoelectric conversion element 209.

This embodiment gives a description on a case of configuring the radiation detection unit 200 in the form of a so-called indirect conversion type which converts incident radiation into an electric charge with use of the above-mentioned phosphor and photoelectric conversion element. However, the radiation detection unit 200 may be configured in, for example, the form of a so-called direct conversion type in which no phosphor is provided and incident radiation is directly converted into an electric charge. The radiation detection unit 200 executes accumulation of electric charges and reading of electric charges by switching the switching element 208 on and off, and a radiographic image can thus be acquired.

In the two-dimensional sensor array 204 of the radiation detection unit 200, a drive line 211 is arranged for every row of pixels and is connected to each pixel 207 in the row, and the pixel 207 is connected to a drive circuit 201 via the drive line 211. The drive circuit 201 applies a switch-on voltage of the TFT to the drive line 211 for one row of pixels 207, to thereby switch on the switching element 208 of each pixel 207 in the one row. Electric charges accumulated in the pixels 207 are thus discharged through a signal line 210 of each pixel 207 to be held in a sample-and-hold circuit 202. The held electric charges output from the pixels are then sequentially read via a multiplexer 203, and amplified by an amplifier 205. The amplified electric charges are converted by an A/D converter 206 into image data having digital values.

In each pixel 207 in a row for which the reading of electric charges has finished, the switching element 208 of each pixel in the row is switched off by application of a switch-off voltage of the TFT from the drive circuit 201 to the drive line 211 associated with the row. Thus, each pixel 207 in the row to which a bias voltage is applied by a power source 212 returns to a state in which accumulation of electric charges is executed. In this manner, the drive circuit 201 sequentially drives and scans rows in the sensor array 204, and electric charges as output from every pixel 207 are ultimately converted into a digital value. Radiographic image data can thus be read. Control of the drive operation, the reading operation, and the like of the detection unit is executed by the drive control unit 102. The image data converted into digital values is stored in the storage unit 109 in FIG. 1.

Referring back to FIG. 1, a mode of the drive control unit 102 in this embodiment is described next. The drive control unit 102 includes a photographing preparation drive control unit 103, a radiographic image acquisition control unit 104, an offset image acquisition control unit 105, and a determination unit 120. The photographing preparation drive control unit 103 causes the radiation detection unit 200 to make preparations so that radiographing by the radiation imaging apparatus 100 is ready. The radiographic image acquisition control unit 104 uses the radiation detection unit 200 to acquire image data to be used in generation of a radiographic image. The offset image acquisition control unit 105 executes, when acquiring an offset image, control for switching an offset image to be acquired. The offset image acquisition control unit 105 includes the determination unit 120, and the determination unit 120 is configured so as to determine, in offset correction processing described later, which offset image is suitable to acquire and use.

The photographing preparation drive control unit 103 applies a voltage that is the same as a voltage used in photographing to the radiation detection unit 200, and concurrently executes periodic reading of electric charges to reset dark charges accumulated in each pixel. The electric charges read in the reset may be handled as non-image data, and holding of those electric charges in the storage unit 109 may be omitted. The radiographic image acquisition control unit 104 executes, for the radiation detection unit 200, drive control that is the same as that of the photographing preparation drive control unit 103, and concurrently causes each pixel in the state of accumulating electric charges to be irradiated with radiation. The radiographic image acquisition control unit 104 uses the radiation irradiation to read image data accumulated in the radiation detection unit 200, and causes the read image data to be stored as a radiographic image in a radiographic image storage portion 110 of the storage unit 109. Execution of this drive control of the radiation detection unit 200 by the radiographic image acquisition control unit 104 in succession enables photographing of radiographic images as a moving image.

The offset image acquisition control unit 105 also executes, for the radiation detection unit 200, drive control that is the same as the drive control by the photographing preparation drive control unit 103, and concurrently reads image data out of the radiation detection unit 200 without radiation irradiation. The read image data is held as an offset image in an offset image storage portion 111 of the storage unit 109.

For a radiographic image acquired from the radiation detection unit 200 in photographing, an offset correction unit 107 of the image processing unit 106 executes offset correction processing with use of an offset image acquired in advance when there is no photographing taking place. The radiographic image processed by the offset correction processing is transferred via the communication control unit 112 to the image control apparatus 500. Although the offset correction processing alone is described here, correction processing for, for example, correction of a missing pixel and gain correction in which gain fluctuations of an amplifier in the radiation detection unit or the like is corrected may be executed.

Those processes of correction processing are not limited to execution in the radiation imaging apparatus 100. For example, the acquired radiographic image and offset image may be transferred to the image control apparatus 500 without being corrected, to receive correction processing in the image control apparatus 500. The offset image used in the offset correction processing may be, for example, an image obtained by processing of reducing noise components through averaging or the like with use of a plurality of acquired offset images.

In this embodiment, a radiographic image acquired under control of the radiographic image acquisition control unit 104 receives offset correction executed with use of an offset image acquired by the offset image acquisition control unit 105. However, targets of offset correction are not limited to images. For example, offset correction may be executed for digital data prior to image generation, or may be executed for data obtained by executing the above-mentioned gain correction or the like on the digital data. Those pieces of data can collectively be referred to as “image data” or “offset-corrected data.”

As photography modes used in photographing a radiographic image, a plurality of photography modes different from one another in, for example, frame rate, are prepared. Normally, a plurality of offset images are acquired for each of those plurality of photography modes, and the offset correction processing is executed with use of an offset image corresponding to a photography mode of interest. In the present disclosure, images relatively close to one another in, for example, frame rate, are grouped into one image group, and share an offset image so that the commonized offset image is used in the offset correction processing for the images in the image group. This reduces the number of offset images to be acquired before execution of radiographing, and enables acquisition of a required number of offset images in a time shorter than in the related art.

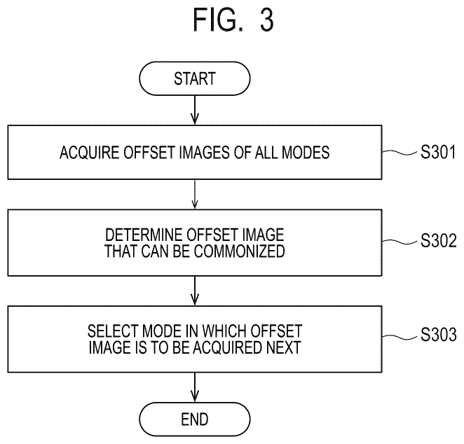

Processing of selecting an offset image that can be commonized is described below with reference to FIG. 3. FIG. 3 is a flow chart for illustrating an example of processing of determining whether offset images acquired in individual photography modes can be commonized. A determination flow illustrated in FIG. 3 is executed when, for example, the radiation imaging apparatus 100 is activated, or when radiographing is executed after a long period of non-use.

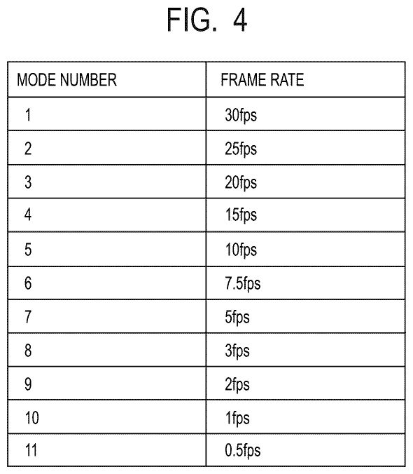

For example, when the radiation imaging apparatus 100 is activated, the determination flow is started by the drive control unit 102. Relationships between photography modes used in the following description and frame rates in the photography modes are shown in FIG. 4. However, a photographing condition that can be varied from photography mode to photography mode is not limited to the frame rate, and every photographing condition set in a photography mode used in normal radiographing can be chosen.

With the start of the determination flow, the offset image update control unit 108 executes, in Step S301, processing of acquiring offset images in all photography modes shown in FIG. 4. The acquired offset images are stored in the offset image storage portion 111 of the storage unit 109. That is, acquisition and storage of offset images are executed for every mode number shown in FIG. 4 in descending order of frame rate, by starting from Mode Number 1, subsequently moving on to Mode Number 2, then to Mode Number 3, and continuing in this manner until Mode Number 11 is reached. When the processing of acquiring offset images in all photography modes is finished, the flow proceeds to Step S302 under control of the drive control unit 102.

In Step S302, determination processing for determining an offset image that can be commonized is executed by the determination unit 120. As a specific example of the determination processing, the determination unit 120 calculates an average value of all pixels from the offset images stored for each mode in Step S301 in the offset image storage portion 111. For example, when a difference in the average value of all pixels between modes is within ±3, values of pixels in the offset images are close to each other, and it is considered that the same offset image is usable in offset correction processing for the different photography modes. It is accordingly determined that an offset correction image to be used can be commonized among different photography modes.

A drop in frame rate prolongs an accumulation time of each pixel, and consequently increases a dark current component. In a case of images close to each other in frame rate, pixels of the respective images are close to each other in accumulation time, and accordingly have average values close to each other. In light of this, the frame rate is also usable as a determination criterion. In the case of the example shown in FIG. 4, for each of three groups of photography modes which are a group including Mode Numbers 1 to 4, a group including Mode Numbers 5 to 8, and a group including Mode Numbers 9 to 11, it is determined that an offset correction image can be commonized. Once an offset image that can be commonized is determined, the flow proceeds to Step S303 under control of the drive control unit 102.

In Step S303, a photography mode of an offset image to be acquired next is selected by the offset image update control unit 108. Specifically, out of each of the three groups for which it is determined in Step S302 that commonization is executable, one photography mode is determined to be a representative. In this case, the photography mode highest in frame rate, for example, may be selected as the representative mode in order to shorten a time spent on acquisition of offset images. The photography modes selected in this case are modes having Mode Numbers 1, 5, and 9. Photographing conditions of Mode Numbers 1, 5, and 9 are accordingly acquisition conditions of offset images to be acquired next. Once acquisition conditions of offset images to be acquired next are determined, the determination flow is ended.

In this embodiment, determination of photography modes for which an offset image can be commonized is executed in Step S302 based on an average value of all pixels of each offset image. However, a condition that is a basis of the determination is not limited to the condition used in the embodiment, and the determination may be made by using, for example, a noise amount and in-plane distribution in addition to an average value of all pixels as conditions. To give an example of using the noise amount as a condition, for photography modes that differ little from one another in noise amount, it is determined that an offset image can be commonized. Specifically, a standard deviation (σ) of pixel values of all pixels is calculated for each offset image in each photography mode, and it is determined that commonization is executable for photography modes that differ from one another in σ by ±5 or less. To give an example of using the in-plane distribution as a condition, a difference between a maximum value and a minimum value of a pixel value in a horizontal or vertical direction running through an image center of an offset image is obtained for each photography mode, and it is determined that commonization is executable for photography modes that differ little from one another in the obtained difference. Specifically, it is determined that commonization is executable for photography modes that differ from one another by, for example, ±15 or less, in terms of a difference (Δ) between the maximum value and the minimum value of the pixel value in a vertical direction running through the image center. Those conditions of the determination may be used separately or may be used in combination.

In this embodiment, a photography mode highest in frame rate is selected in Step S303 as a representative mode. However, the method of selecting a representative mode is not limited to the method used in the embodiment. For example, a photography mode smallest in noise amount may be selected, and a photography mode having a frame rate closest to a median value in the group may be selected as a representative mode. In a case in which a minimum value of pixel values in a radiographic image is smaller than a minimum value of pixel values in an offset image, there is a possibility that the subtraction in the offset correction processing renders the pixel value into a negative value. In such a case, a mode smallest in average value of all pixels may be selected as a representative mode.

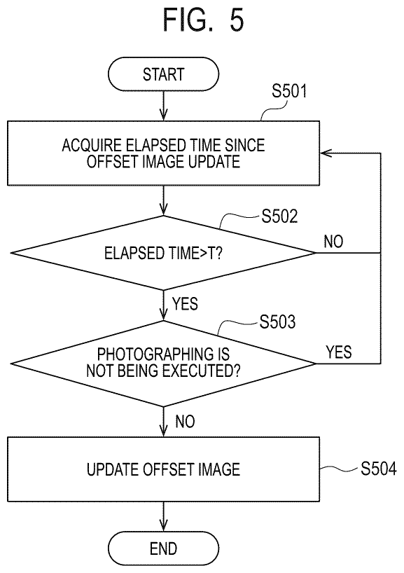

Offset image update processing executed by the offset image update control unit 108 is described next with reference to FIG. 5. FIG. 5 is a flow chart for illustrating offset image update processing in this embodiment. For example, when a user inputs, via the operation UI 407, an instruction to start photographing of a radiographic image, the offset image update control unit 108 starts the offset image update processing, and the flow proceeds to Step S501. Simultaneously, the photographing preparation drive control unit 103 executes preparation operation of the radiation detection unit 200 to ready the radiation imaging apparatus 100 for radiographing.

In Step S501, an elapsed time since the offset image update processing executed last is acquired with use of the internal clock 113. In a case in which offset image update has not been executed in the immediate past even once, for example, when the offset image update processing to be executed is first processing of the day, the elapsed time is set to a maximum value. Once the elapsed time is acquired, the flow proceeds to Step S502 under control of the offset image update control unit 108.

In Step S502, the offset image update control unit 108 compares the elapsed time acquired in Step S501 and a threshold elapsed time T determined in advance. When it is determined that the elapsed time >T is satisfied, the flow proceeds to Step S503. In a case in which the elapsed time ≤T is satisfied, the flow returns to Step S501. In Step S501, the preparation operation by the photographing preparation drive control unit 103 is continued, and the processing of acquiring the elapsed time is repeated. In this embodiment, T is five minutes as an example of the threshold elapsed time.

In Step S503, the offset image update control unit 108 checks whether the radiation imaging apparatus 100 is executing fluoroscopic or photographing processing. When it is confirmed that no fluoroscopic or photographing processing is being executed, the flow proceeds to Step S504. When it is confirmed that fluoroscopic or photographing processing is being executed, the flow returns to Step S501. When fluoroscopic or photographing processing is being executed, the acquisition of the elapsed time is repeated in Step S501.

In Step S504, the preparation operation of the photographing preparation drive control unit 103 is switched to offset image acquisition processing of the offset image acquisition control unit 105, and the offset image update processing is executed. Specifically, update of offset images is executed sequentially for Mode Numbers 1, 5, and 9 selected in Step S303 of FIG. 3. Images acquired by the radiographic image photographing processing confirmed in Step S503 to be in the middle of being executed can be corrected by offset correction processing that uses offset images updated in Step S504. After the update of offset images, the flow proceeds further and the offset image update processing is ended.

A specific example is given with reference to FIG. 4 on offset correction processing of a radiographic image acquired from the radiation detection unit 200. For example, photography modes identified by Mode Number 4 and Mode Number 1 are included in a photography mode group for which a shared offset image is usable, and it is assumed that an offset image acquired under the condition of Mode Number 1 can be used in offset correction. In this case, when a radiographic image is to be photographed in a mode identified by Mode Number 4, an already acquired offset image of Mode Number 1 which is stored in the offset image storage portion 111 is used in offset correction processing of a radiographic image acquired from the radiation detection unit 200. After the offset correction processing is executed by the offset correction unit 107 of the image processing unit 106 with the use of the offset image of Mode Number 1, the corrected radiographic image is transferred through the communication control unit 112 to the image control apparatus 500.

As described above, in the embodiment according to the present disclosure, an offset image that can be used in a plurality of photography modes in a shared manner is selected, and only offset images of a minimum number of photography modes for which commonization is executable are updated. Then, with respect to a radiographic image of a photography mode for which it is determined that commonization is executable, a selected offset image alone is updated and offset correction processing is executed for the radiographic image with use of the updated offset image. Execution of offset correction processing in this mode enables shortening of a time required to update offset images, compared to a mode of the related art in which offset images of all photography modes are updated. As a result, radiographing using the latest offset information can be executed immediately.

When the offset image update processing is executed right after photographing, there is a possibility that a residual image is included in the offset image. Accordingly, an elapsed time since completion of photographing may be measured so that the offset image update processing is executed when a condition about the elapsed time since completion of photographing and a condition about the elapsed time since the offset image update processing are both satisfied.

### Modification Example

In the embodiment described above, a mode in which the determination unit 120 is provided inside the radiation imaging apparatus 100 is described. However, modes of the present disclosure are not limited to the mode of the embodiment. To give an example with reference to FIG. 4, when a radiographic image is acquired under the condition of Mode Number 4 by the radiation imaging apparatus 100 of the present disclosure, there may be a case in which the offset correction image of Mode Number 4 has not been updated and a suitable offset correction image has not been stored in the immediate past. In such a case, the image processing unit 106 or the image control apparatus 500, for example, can execute offset correction processing with use of, not an offset image of the mode identified by Mode Number 4, but an offset image of the mode identified by Mode Number 1 which is determined to be suitable for the use of an offset image shared with the mode identified by Mode Number 4. In this case, the present disclosure is configurable as an image processing apparatus.

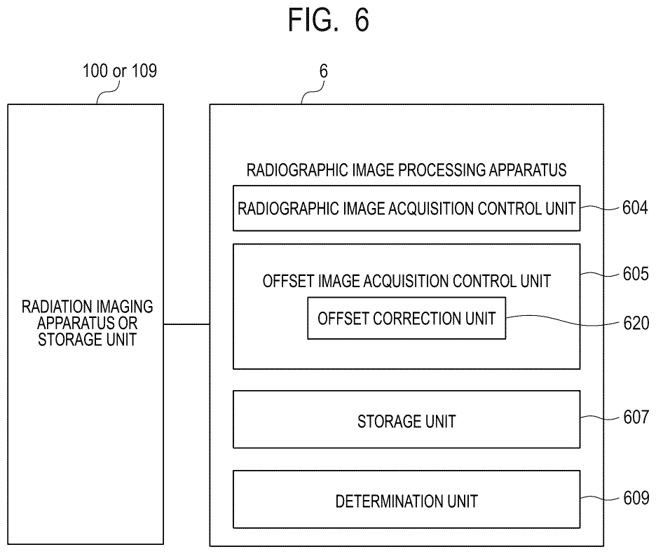

One embodiment of the present disclosure as described above is described below as a modification example with reference to FIG. 6. FIG. 6 is a diagram for illustrating a configuration example of a radiographic image processing apparatus 6 according to the modification example of the present disclosure. The radiographic image processing apparatus 6 illustrated in FIG. 6 includes a radiographic image acquisition control unit 604, an offset image acquisition control unit 605, an offset correction unit 607, and a storage unit 609. The radiographic image processing apparatus 6 is connected to the radiation imaging apparatus 100, or to the storage unit 109 in which images photographed by the radiation imaging apparatus 100 are stored.

The radiographic image acquisition control unit 604 acquires a radiographic image from the radiation imaging apparatus 100 or the storage unit 109. The offset image acquisition control unit 605 acquires an offset image from the radiation imaging apparatus 100 or the storage unit 109. A determination unit 620 included in the offset image acquisition control unit 605 is capable of executing the processing illustrated in FIG. 3, as is the determination unit 120 described in the embodiment. That is, the determination unit 620 determines photography modes for which a shared offset image is usable in offset processing, and selects an offset image to be used in offset correction of those photography modes. The offset correction unit 607 executes offset correction on the radiographic image acquired by the radiographic image acquisition control unit 604 with use of the offset image acquired by the offset image acquisition control unit 605. The storage unit 609 stores photography modes determined by the determination unit 620 to be suitable for shared use of an offset image with one another, in association with a selected offset correction image.

In a case in which an offset image corresponding to the acquired radiographic image has been acquired in the immediate past, the offset correction unit 607 executes offset correction of the radiographic image with use of this offset image. However, there may be a case in which an offset image corresponding to a photography mode for acquiring an image cannot be acquired, for example, a case in which a time to acquire offset images is continuous or radiographic images are photographed in succession. Even in such cases, selected offset images are updated in the radiation imaging apparatus 100 described as the embodiment. The offset correction unit 607 executes offset correction of photography modes determined to be suitable for the shared use of an offset image with one another, with use of an offset image updated in a photography mode that has been used when a selected offset image stored in the storage unit 609 has been acquired. This processing enables the image processing apparatus according to this modification example to execute offset correction of an acquired radiographic image with use of an appropriate offset image even when an offset image corresponding to a radiographic mode of interest is not present.

As described above, the radiation imaging apparatus 100 according to one embodiment of the present disclosure includes an image data acquisition unit (the radiographic image acquisition control unit 104) and an offset data acquisition unit (the offset image acquisition control unit 105). The radiation imaging apparatus 100 further includes the offset correction unit 107, the determination unit 120, and an update unit (the offset image update control unit 108). The radiographic image acquisition control unit 104 functions as an example of the image data acquisition unit which acquires, under a photographing condition of a photography mode selected from a plurality of photography modes for acquiring radiographic images of a plurality of types, radiographic image data (a radiographic image) related to the selected photography mode. The offset image acquisition control unit 105 functions as an example of the offset data acquisition unit which acquires, for each of the plurality of photography modes, offset correction data (an offset image) acquired in the photography mode. The offset correction unit 107 functions as an example of an offset correction unit which corrects the radiographic image with use of the acquired offset image. The determination unit 120 determines photography modes for which a shared offset image is usable, out of the plurality of photography modes. The determination unit 120 also functions as an example of a determination unit which selects, out of offset images corresponding to the respective determined photography modes, one offset image to be used by the offset correction unit 107 in correction of the determined photography modes. The offset image update control unit 108 functions as an example of the update unit which updates the one offset image to be used for the photography modes determined by the determination unit 120 to be suitable for the shared use of an offset image with one another. To describe in more detail, the offset image update control unit 108 causes the offset image acquisition control unit 105 to acquire an offset image in the same photography mode as a photography mode in which the offset image selected by the determination unit 120 has been acquired, so that the acquired offset image corresponds to the selected offset image. This enables efficient acquisition of latest offset correction data in a short time even when the number of radiography modes that may be used is large, and execution of appropriate offset correction is accordingly achieved.

In the radiation imaging apparatus 100 described above, the determination unit 120 can determine photography modes for which a shared offset image is usable, based on an average value of pixel values of all pixels in an offset image which is an average value of the offset correction data. Alternatively, the determination unit 120 may determine photography modes for which a shared offset image is usable, based on noise amounts of all pixels in an offset image. The determination unit 120 may also determine photography modes for which a shared offset image is usable, based on in-plane distribution of pixel values of all pixels which is in-plane distribution of an offset image.

The determination unit 120 may select, out of offset images to be used for the photography modes determined to be suitable for shared use of the common offset images, an offset image that corresponds to a photography mode highest in frame rate. Alternatively, the determination unit 120 may select, out of offset images to be used for the photography modes determined to be suitable for shared use of the common offset image, an offset image that corresponds to a photography mode smallest in noise amount. Further, the determination unit 120 may select, out of offset images to be used for the photography modes determined to be suitable for shared use of the common offset image, an offset image that corresponds to a photography mode smallest in average value of pixel values of all pixels.

The present disclosure is also applicable to the radiation imaging system 1. In this case, the radiation imaging system 1 includes the radiation generation apparatus 300 which generates radiation; the control apparatus 400 which controls the radiation generation apparatus 300, and the above-mentioned radiation imaging apparatus 100.

An image processing apparatus is also configurable from the present disclosure. In the image processing apparatus, offset correction is executable on an image acquired in one photography mode even when, for example, there is an offset correction image that can be used in a shared manner by a group of photography modes including the one photography mode, but there is no offset correction image of the one photography mode itself. Such an image processing apparatus (the image control apparatus 500) can include an image data acquisition unit, an offset correction data acquisition unit, and an offset correction unit. The image data acquisition unit has, as an example, a function of acquiring radiographic image data acquired in each of a plurality of photography modes which is similar to the function of the radiographic image acquisition control unit 104. The offset correction data acquisition unit has, as an example, a function of acquiring offset correction data corresponding to each of the plurality of photography modes, which is similar to the function of the offset image acquisition control unit 105. The offset correction unit can use one piece of offset correction data selected out of pieces of offset correction data that correspond to respective photography modes for which shared offset correction data is usable. With the use of the one piece of offset correction data, the offset correction unit executes offset correction on pieces of radiographic image data acquired in the respective photographic modes for which the shared offset correction data is usable.

### Other Embodiments

Embodiment(s) of the present disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

This application claims the benefit of Japanese Patent Application No. 2023-175218, filed Oct. 10, 2023, which is hereby incorporated by reference herein in its entirety.

What is claimed is:

1. A radiation imaging apparatus comprising:
an image data acquisition unit configured to acquire radiographic image data that is related to a photography mode selected from a plurality of photography modes for acquiring radiographic images of a plurality of types;
an offset correction data acquisition unit configured to acquire offset correction data corresponding to each of the plurality of photography modes;
an offset correction unit configured to correct the radiographic image data with use of the acquired offset correction data;
a determination unit configured to determine, out of the plurality of photography modes, photography modes for which common offset correction data shared with one another is usable, and select, out of pieces of offset correction data corresponding to the respective determined photography modes, a piece of offset correction data to be used by the offset correction unit in correction of the determined photography modes; and
an update unit configured to control the offset correction data acquisition unit so that, in order to update offset correction data to be used for the photography modes determined to be suitable for shared use of the common offset correction data, offset correction data corresponding to the selected offset correction data is acquired.

2. The radiation imaging apparatus according to claim 1, wherein the determination unit is configured to determine the photography modes for which the common offset correction data shared with one another is usable, based on an average value of the offset correction data.

3. The radiation imaging apparatus according to claim 1, wherein the determination unit is configured to determine the photography modes for which the common offset correction data shared with one another is usable, based on a noise amount of the offset correction data.

4. The radiation imaging apparatus according to claim 1, wherein the determination unit is configured to determine the photography modes for which the common offset correction data shared with one another is usable, based on in-plane distribution of the offset correction data.

5. The radiation imaging apparatus according to claim 1, wherein the determination unit is configured to select, out of pieces of offset correction data to be used for the photography modes determined to be suitable for shared use of the common offset correction data, a piece of offset correction data that corresponds to a photography mode highest in frame rate.

6. The radiation imaging apparatus according to claim 1, wherein the determination unit is configured to select, out of pieces of offset correction data to be used for the photography modes determined to be suitable for shared use of the common offset correction data, a piece of offset correction data that corresponds to a photography mode smallest in noise amount of the offset correction data.

7. The radiation imaging apparatus according to claim 1, wherein the determination unit is configured to select, out of pieces of offset correction data to be used for the photography modes determined to be suitable for shared use of the common offset correction data, a piece of offset correction data that corresponds to a photography mode smallest in average value of the offset correction data.

8. A radiation imaging system comprising:
a radiation generation apparatus configured to generate radiation;
a control apparatus configured to control the radiation generation apparatus; and
the radiation imaging apparatus according to claim 1.

9. A radiographic image processing apparatus comprising:
an image data acquisition unit configured to acquire radiographic image data acquired in each of a plurality of photography modes;
an offset correction data acquisition unit configured to acquire offset correction data corresponding to each of the plurality of photography modes; and
an offset correction unit configured to execute offset correction on pieces of radiographic image data acquired in respective photography modes which are included among the plurality of photography modes and for which common offset correction data shared with one another is usable, with use of one piece of offset correction data selected from pieces of offset correction data corresponding to the respective photography modes for which the common offset correction data shared with one another is usable.

10. A method of controlling a radiation imaging apparatus, the method comprising:
acquiring radiographic image data that is related to a photography mode selected from a plurality of photography modes for acquiring radiographic images of a plurality of types;
acquiring offset correction data corresponding to each of the plurality of photography modes;
correcting the radiographic image data with use of the acquired offset correction data;
determining, out of the plurality of photography modes, photography modes for which common offset correction data shared with one another is usable, and selecting, out of pieces of offset correction data corresponding to the determined photography modes, a piece of offset correction data to be used in correction of the determined photography modes; and
acquiring, in order to update offset correction data to be used for the photography modes determined to be suitable for shared use of the common offset correction data, offset correction data corresponding to the selected offset correction data.

11. A non-transitory tangible medium having recorded thereon a program for causing a computer to perform steps of the method of controlling a radiation imaging apparatus according to claim 10.
